(19) United States
(12) Patent Application Publication
(10) Pub. No.: US 2012/0202212 A1
(43) Pub. Date: Aug. 9, 2012

(54) METHODS FOR GENETIC COMPOSITION ANALYSIS OF NATURAL PRODUCTS

(76) Inventor: Danica Taylor Harbaugh Reynaud, Elcerrito, CA (US)

(21) Appl. No.: 13/369,585

(22) Filed: Feb. 9, 2012

Related U.S. Application Data
(60) Provisional application No. 61/441,241, filed on Feb. 9, 2011.

(51) Int. Cl. C12Q 1/68

(52) U.S. Cl. 435/6.12

(57) ABSTRACT

Provided herein are novel methods for identifying component species in a natural product using genetic testing methods.

### RELATED APPLICATIONS

The present application claims the benefit of U.S. Provisional Application No. 61/441,241, filed Feb. 9, 2011, the disclosure of which is incorporated by reference herein in its entirety, including drawings.

### BACKGROUND

Worldwide over 4 billion people use biologically-derived or natural products, including supplements, pharmaceuticals and traditional medicine. Many more use cosmetics, food and beverages that contain natural herbs and spices. In the US alone, there are approximately $100 billion in sales annually of nutritional supplements, with the cosmetics and food industries accounting for significantly more.

Raw materials (e.g., dried botanical, fungal, bacterial, and animal species) and extracts used to formulate natural products often come in processed forms and are subject to both inadvertent and intentional substitution, adulteration, and contamination. Although some adulterants can be chemically inert and harmless, others may pose extreme health threats to people who consume them. Therefore, it is critical to develop scientifically valid authentication methods that can address these issues to ensure the safety, quality, and efficacy of natural products. Such authentication methods should ideally identify the taxon of the primary and any contaminating species present in the product based on verified reference vouchers.

A number of morphological and chemical approaches for the authentication of medicinal herb species have been disclosed previously. However, many of these methods are limited in their ability to differentiate between closely related species, identify ground or processed materials, or detect fillers (e.g., soy powder) and allergenic species (e.g., peanuts). Such limitations have led to the development of methods that utilize DNA sequence data.

The use of DNA sequencing methods for botanical identity testing provides several advantages over morphological and chemical methods. For example, DNA sequencing methods can be performed on degraded, powdered, and processed plant material from any plant part, as well as on mixtures thereof. DNA sequencing methods can also differentiate between closely related taxa, populations, and even individuals.

A number of techniques for DNA-based sequence analysis are known in the art, including AFLPs, RFLPs, PCR, diagnostic PCR, ARMS, RAPD, SCAR, SSR, SSCP, and microarrays. However, many of these techniques are unsuitable for authenticating the contents of natural products. AFLPs, RFLPs, and many PCR methods rely on a priori knowledge of the contents of a material and specific primers, so they are not useful for identifying unexpected adulterants. Many of the other techniques require large up-front costs, are time consuming to develop and perform, or cannot be run on a large-scale. Some DNA-based methods do not include comparison of data to authenticated reference sequences for taxonomic identifications, including multiple reference materials for both primary and contaminating species. Many DNA-based methods are unsuitable for processed, mixed, or complex materials, including but not limited to finished dietary supplements, foods, and herb blends. Other DNA-based methods may not account for degradation or fragmentation of materials, or secondary-compounds which are commonly present in medicinal herbs, especially degraded ones. As summarized by Teletchea 2005, “simultaneous detection of several species is certainly one of the greatest challenges in the field [food], but still remains unresolved.”

Given the disadvantages of previously developed DNA-based analysis methods, there is a need in the art for more effective methods for analyzing the composition of natural products using DNA sequence analysis.

### SUMMARY

In certain embodiments, methods are provided for identifying one or more component species in a natural product by isolating genomic DNA from a natural product test sample and amplifying one or more target regions within the genomic DNA, followed by sequencing the one or more target regions and comparing the resultant sequences to one or more reference sequences. In certain embodiments, component species sequences are classified as primary or contaminating species sequences. In certain embodiments, the component species sequences are further subjected to single nucleotide polymorphism (SNP) analysis and/or phylogenetic analysis.

In certain embodiments, methods are provided for identifying one or more component species in a natural product by isolating genomic DNA from a natural product test sample, amplifying one or more target regions within the genomic DNA, separating the amplified target regions into multiple DNA templates by cloning the multiple DNA templates into bacterial host cells, sequencing the multiple DNA templates from positive bacterial clones, and comparing these sequences to one or more reference sequences. In certain embodiments, component species sequences are classified as primary or contaminating species sequences. In certain embodiments, the component species sequences are further subjected to SNP analysis and/or phylogenetic analysis.

In certain embodiments, methods are provided for identifying one or more component species in a natural product by isolating genomic DNA from a natural product test sample, amplifying one or more target regions within the genomic DNA, performing simultaneous sequencing (e.g., next-generation sequencing) on the amplified target regions, and comparing the resultant sequences to one or more reference sequences. In certain embodiments, component species sequences are classified as primary or contaminating species sequences. In certain embodiments, the component species sequences are further subjected to SNP analysis and/or phylogenetic analysis.

### BRIEF DESCRIPTION OF DRAWINGS

FIG. 1: Image of the electropharogram of the ITS DNA sequence of Schisandra chinensis after cloning.

FIG. 2: Image of the electropharogram of the ITS DNA sequence of S. chinensis prior to cloning.

FIG. 3: Aligned matrix of DNA sequence data from S. chinensis and related species illustrating SNPs. The basepair “C” at position 100 is only present in S. chinensis and distinguishes it from the other species; therefore, this is considered a SNP.

FIG. 4: Phylogeny, or branching diagram, of S. chinensis ITS DNA sequence data and closely related species illustrating identification using phylogenetic analysis. The numbers above the branches represent the Maximum Likelihood support values (out of 100).

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The difficulties associated with accurate identification of both primary and contaminating species, and the failure of previously developed methods to overcome these difficulties, is well known in the art. For example, Yip 2007 acknowledges that contamination by non-target DNA such as bacteria, fungi, or insects (but also plants) may pose a problem, and therefore recommends species-specific methods. However, these methods do not allow for the detection of unexpected contaminants. Zhang 2007 and Yip 2007 both acknowledge that PCR inhibitors and secondary compounds such as those often found in medicinal plants can make PCR and DNA sequencing methods difficult, with Zhang stating that it is necessary to “have a more effective, accurate, reliable, and sensitive technology for the authentication of herbs . . . [p]articularly, Chinese formulations noteworthy of multiple plant components make the identification more difficult, but it is not impossible. Testing for unknown contaminants is extremely difficult.”

Provided herein in certain embodiments are methods for identifying the component species in a natural product by genetic analysis. As set forth in the examples below, these methods were used to successfully identify both primary and contaminating species in natural products. The methods disclosed herein provide several advantages over previously developed methods: 1) they allow for accurate, repeatable identification of all component species within a natural product, including contaminating species, rather than identification of a single component species; 2) unlike previously developed methods that utilize microarrays or species-specific PCR, they do not require costly and time-consuming up-front development; 3) they allow for quantification or semi-quantification of all taxa present in a natural product; 4) they can be carried out using a relatively small amount of starting material; 5) incorporation of a DNA purification step prior to PCR eliminates issues associated with PCR inhibitors and secondary compounds.

The term “natural product” as used herein refers to any composition comprising one or more components derived from plant, animal, fungal, or bacterial sources. The components in a single natural product may be derived from multiple sources. For example, a single natural product may comprise components from two or more primary species such as two or more plant species, or components from one or more primary species and one or more contaminating species. Components in a natural product may be derived from any part of a plant, animal, fungal, or bacterial source, including both whole organisms and portions thereof in a fresh, dried, liquid, or frozen state. Natural products may include raw materials or extracts derived from water or solvent extraction. Alternatively, natural products may be in a finished or processed form, including for example in the form of capsules, pills, or food products. Examples of natural products that may be analyzed include herbal supplements, botanically-derived pharmaceuticals, traditional medicine products, skin care products and other cosmetics products, foods, beverages, and components thereof, including the ingredients used to formulate the products.

The term “component species” as used herein with regard to natural products refers to all species present in the natural product.

The term “primary species” as used herein with regard to natural products refers to any species that is expected to occur in a particular natural product. A single natural product may contain more than one primary species. For example, a primary species may include any species that is listed on the labeling for a particular natural product.

The term “contaminating species” as used herein with regard to natural products refers to any species in a natural product that is not expected to occur in that particular natural product. A contaminating species may have been introduced into a natural produce either intentionally or inadvertently.

The term “species” as used herein with regard to component species, primary species, and contaminating species may refer to any rank of interest. For example, species may refer to the formal taxonomic rank of species. Alternatively, species as used in this context may refer to another taxonomic rank (e.g., family, genus), or to a sub-specific taxon such as variety, strain, subspecies, lineage, or population.

In certain embodiments of the methods provided herein, genomic DNA (which may include nuclear, ribosomal chloroplast, and/or mitochondrial DNA, as well as RNA) from a natural product test sample is isolated and purified, and a target region is amplified using PCR or any other method appropriate to the downstream sequencing method being used (e.g., for next-generation sequencing). The amplified target regions are sequenced, and the sequences are compared to determine whether sequences from multiple component species are present. In certain embodiments, the resultant sequences are compared to one or more reference sequences in order to identify component species. In addition to sequencing, amplified target regions may undergo additional analyses, for example to confirm the success of the amplification reaction. In those embodiments where direct sequence analysis indicates the presence of more than one component species, the amplified target regions may be separated into multiple DNA templates, where each DNA template comprises a target region from a single strand of DNA derived from a particular component species. In certain embodiments, the multiple DNA templates may be cloned into one or more bacterial host cells. The cloned multiple DNA templates are sequenced, and the component species serving as the source for each DNA sequence is determined based on comparisons to one or more reference sequences. In other embodiments, cloning is replaced by simultaneous sequencing of multiple DNA templates, followed by comparison of the resultant sequences to one or more reference sequences. In certain of these embodiments, the multiple DNA templates are not separated prior to sequencing. In certain embodiments, DNA template sequences (whether from clones or from simultaneous sequencing) may undergo additional analysis, including examination of SNPs and/or phylogenetic analysis.

Isolation of genomic DNA from a natural product test sample may be carried out using any technique known in the art. For example, isolation of genomic DNA may be done using cetyl trimethylammonium bromide (CTAB) and a variety of commercially available kits that use spin columns, vacuums, and/or magnetic beads (e.g., DNEasy Plant Kit, DNEasy Blood and Tissue Kit, QIAamp Stool Kit; Qiagen Inc.). In certain embodiments, DNA isolation is performed manually, while in embodiments it may be automated.

In certain embodiments, genomic DNA may be purified prior to amplification to remove inhibitors and secondary compounds using methods known in the art. For example, genomic DNA may be purified by phenol/chloroform extraction and/or ethanol precipitation, commercially available kits that use technologies such as silica spin-columns, vacuums, and/or magnetic beads (e.g., MinElute PCR Purification Kit, QIAquick PCR Purification Kit; Qiagen Inc.), or enzymes (e.g., EXO-SAP; USB Corp). In certain embodiments, DNA purification is performed manually, while in others it is automated.

Amplification of a target region in a test sample can be carried out using any method known in the art that generates amplification product appropriate for the downstream sequencing application. In certain embodiments, amplification may be carried out using standard PCR techniques, including in certain embodiments emulsion PCR or multiplex PCR. In those embodiments where multiplex PCR is utilized, a unique tag is attached to each primer before PCR amplification.

Primers for use in amplification of target regions may vary in length. In certain embodiments, they may be between 10 and 30 basepairs in length. In certain embodiments, a single target region is amplified from a test sample. In these embodiments, amplification of the single target region may be repeated two or more times. In other embodiments, multiple target regions may be amplified from a single test sample. In these embodiments, the multiple amplifications can be performed in a single amplification reaction (i.e., utilizing multiple primer sets in a single amplification reaction) or in multiple amplification reactions.

In certain embodiments, a target region is about 30 to about 2000 basepairs in length. In certain embodiments, the target region comprises one or more genes or portions thereof. Examples of genes or portions thereof that may be present at a target region include Internal Transcribed Spacer (ITS), ITS1, ITS2, matK, 3 'trnK, rbcL, psbA-trnH intergenic spacer, cox1, cox2, 16S, COI, External Transcribed Spacer (ETS), waxy, 18S, 5S, atpB, atpB-rbcL, adh, GPAT, nadF, rpl16, rps16, rps4, trnL-trnF, nad, trnL intron, and trnl-trnF intergenic spacer regions. In certain embodiments, a target region may comprise a non-coding sequence in addition to or lieu of coding gene sequences.

In certain embodiments, primers used for amplification of target regions are taxon-specific, meaning that they are capable of amplifying a target region from all organisms falling within a targeted subset of organisms (e.g., all organisms falling within a particular taxonomic family, genus, or species). In other embodiments, the primers are universal, meaning that they are capable of amplifying a target region from all organisms present in a test sample. Target regions are preferably sufficiently variable between species to allow for differentiation of sequences from all potential primary and contaminating species.

In certain embodiments, amplified target region DNA may be purified using methods known in the art. For example, amplified DNA may be purified by phenol/chloroform extraction and/or ethanol precipitation, commercially available kits that use technologies such as silica spin-columns, vacuums, and/or magnetic beads (e.g., MinElute PCR Purification Kit, QIAquick PCR Purification Kit; Qiagen Inc.), or enzymes (e.g., EXO-SAP; USB Corp). In certain embodiments, DNA purification is performed manually, while in others it is automated.

In those embodiments where the amplification reaction is visualized to confirm the success of the amplification reaction, visualization may be accomplished using any method known in the art. For example, amplified DNA may be visualized by staining with ethidium bromide, SYBR-Safe® (Invitrogen, Inc.), or another suitable DNA stain, running through an agarose gel, E-Gel® (Invitrogen, Inc.), or similar apparatus using an electric current, and visualizing using an appropriate light source. In some embodiments, the total amount of DNA may be determined using a quantitative DNA ladder run on the gel, a spectrophotometer (e.g., NanoDrop®, Thermo Scientific, Inc.), a quantitative PCR machine, or another instrument designed to determine concentrations of DNA.

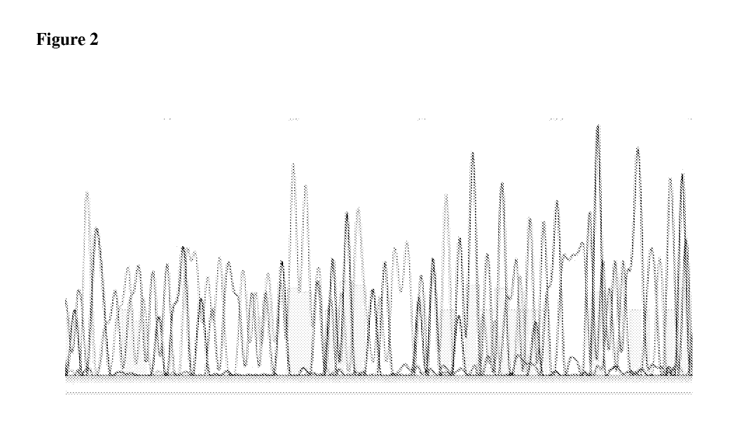

In certain embodiments, separation and cloning of multiple DNA templates is carried out regardless of any sequencing results with the amplified target region. In other embodiments, separation and cloning is only conducted where the DNA sequences from the amplified targeted sequences demonstrate multiple-overlapping signals (i.e., where sequences from multiple component species are indicated as in FIG. 2).

In those embodiments where the multiple DNA templates are separated and cloned into a bacterial host cell, cloning may be performed using any method known in the art. In certain embodiments, bacterial cells transformed with the multiple DNA templates undergo selection to identity clones containing a DNA template sequence. DNA from positive clones is then sequenced, and the resultant sequences are compared to one or more reference sequences. Sequences may be obtained from 1 to 10 colonies, 10 to 200 colonies, or more than 200 colonies from a single sample. In certain preferred embodiments, both the 5′ and 3′ DNA strands are sequenced.

The use of cloning to identify component in raw materials or natural products has previously been discouraged in the art. For example, Lum 2006 states “[c]loning the PCR product and sequencing the clones is one way to sample the composition of a PCR product that may be derived from multiple species, but it is difficult to know how many clones are representative of the diversity found in the sample. Also, cloning numerous PCR fragments is time-consuming and expensive, and unlikely to be a cost-effective way for a rapid analysis of botanicals and their contaminants/adulterants.” Pun 2009 argued that techniques such as cloning and sequencing offer a potentially reliable solution for identifying multiple species in a complex mixture, but noted that they have not been used extensively for species identification and that they involve long development times and high costs. Teletchea 2008 sampled 15-20 clones per product, and concluded that the method was not accurate in identifying more than a couple of species and recommended the use of microarrays instead. Therefore, the finding in the present application that a sequencing method that utilizes a cloning step can be used to efficiently and accurately classify multiple component species within a natural product sample was entirely unexpected.

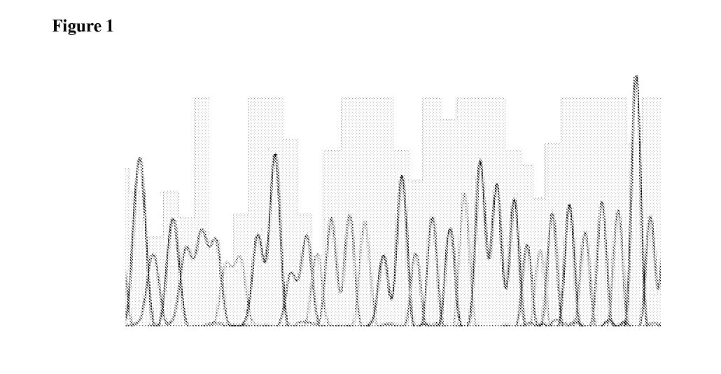

Sequencing of PCR products and/or cloned DNA templates may be carried out using any technique known in the art. For example, sequencing may be carried out using Sanger sequencing. “Sanger sequencing” as used herein refers to any sequencing technique that utilizes dideoxy chain technology. In certain embodiments, a positive identification can be made if the sequence is free of highly overlapping signals (FIG. 1). In certain preferred embodiments, both the 5′ and 3′ DNA strands are sequenced. Alternatively, parallel sequencing may be carried out using next generation sequencing techniques, with or without a prior cloning step. “Next-generation sequencing techniques” as used herein refers to sequencing techniques that do not fall within the scope of Sanger sequencing, including for example Solexa (Illumina), Ion Torrent (Life Technologies), SOliD (Applied Biosystems), 454 Pyrosequencing (based on the detection of released pyrophosphate (PPI)), or any other non-Sanger sequencing methods previously developed or developed in the future.

In certain embodiments, reference sequences for use in identifying component species are derived from authenticated materials from a whole organism or a portion of an organism. In certain of these embodiments, the standard is derived from vouchered materials, while in others it is obtained from publicly available sources or databases. In certain embodiments the reference sequences used for comparison have been authenticated using one or more of the following characteristics: morphological genetic characters, chemical, UV, near-infrared, or using any other method known in the art of organism identification or characterization. In certain embodiments, only sequences from primary species are specifically identified. In these embodiments, all reference sequences correspond to sequences from a primary species. Sequences that do not match the primary species reference sequences are categorized as contaminating species without being specifically identified. In other embodiments, contaminating species are specifically identified. In these embodiments, the reference sequences include one or more sequences from species other than the primary species, where these additional sequences correspond to one or more contaminating species.

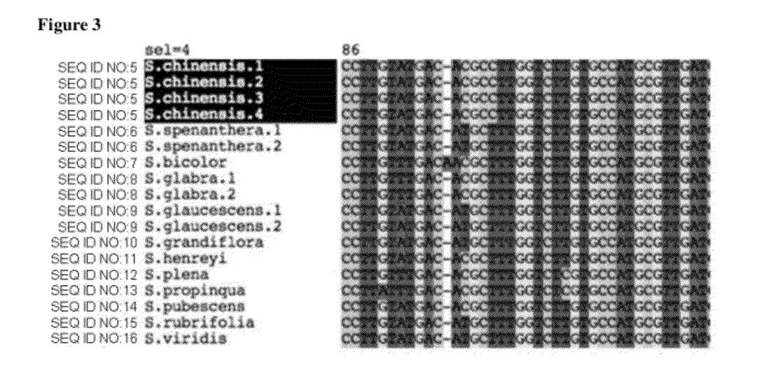

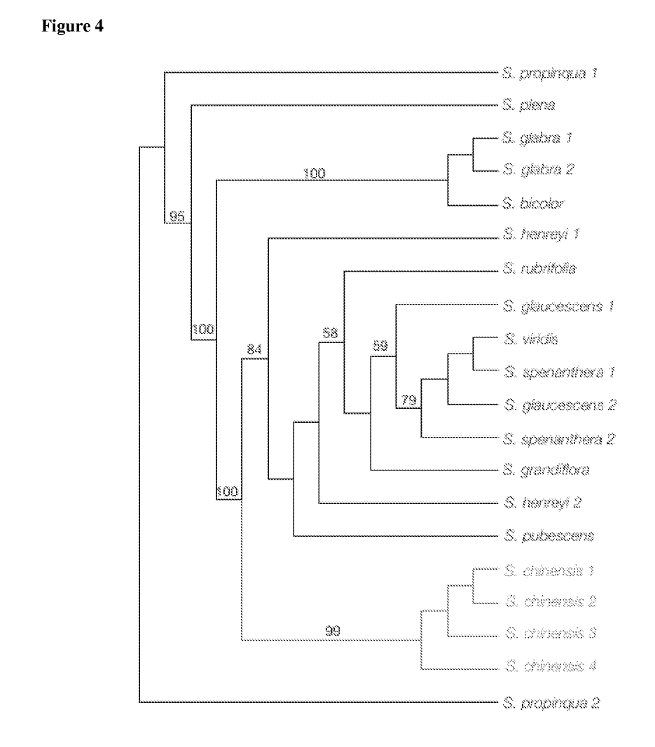

In certain embodiments, the identity of a component species sequence is determined by comparing the sequence in an aligned matrix, visually, using an algorithm, by SNP comparisons (FIG. 3), by producing a phylogenetic or cladistic analysis (FIG. 4), or by other methods known in the art of DNA sequence comparison. There are a number of methods well known in the art for creating a phylogeny using DNA sequences, including but not limited to neighbor joining (NJ), distance, maximum parsimony (MP), maximum likelihood (ML), and Bayesian inference. The parameters used for each analysis may vary slightly due to the size and complexity of the dataset. A variety of computer programs and algorithms known in the art may be used to analyze and align DNA sequence data and to build phylogenetic trees. In certain embodiments, these algorithms will provide support measures on the branches of the phylogeny. A phylogeny is considered robust if there are high support values in some embodiments with a value at least of 50%. In certain embodiments, a sequence from an unknown component species can by identified by its placement in the phylogeny relative to the DNA sequences of known specimens. In certain embodiments, a sample is construed to contain a species X if it exhibits at least 50%, at least 75%, at least 80%, at least 85%, at least 90%, at least 95%, at least 99%, or 100% identity similarity to known reference sequences.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Genetic Analysis of Alleged Schisandra Powder Product

Genomic DNA Isolation

Total genomic DNA from a schisandra powder (Schisandra chinensis) capsule produced by company A was extracted using the DNEasy Plant Mini Kit (Qiagen). The extraction was performed using an automated QIAcube machine (Qiagen) running the standard protocol for the DNEasy Plant Mini Kit. Using sterile techniques, a total of 20 μl of genomic DNA was transferred using a pipet to a new sterile microcentrifuge tube and placed back into the QIAcube. The genomic DNA was purified using the MinElute PCR Purification Kit (Qiagen) with standard protocol.

PCR Amplification

The ITS region was selected for amplification because it can be used to identify distantly related species, including for example Fabaceae and Schisandraceae, as well as to differentiate between closely related species. PCR was carried out using the forward primer ITS5 (5′-GGAAGTAAAAGTCGTAACAAGG-3′) and the reverse primer is ITS4 (5′-TCCTCCGCTTATTGATATGC-3′) (White 1990). 1 μl of purified genomic DNA was amplified in a 20 μl total volume reaction using a Bioneer Hot-Start AccuPowder Pre-Mix Tube (containing buffer; MgCl₂; deoxyribonucleotides dATP, dTTP, dGTP, dCTP) with 0.75 μl of a 10 μM solution of both the forward and reverse primers. The PCR reaction was placed in a thermal cycler with the following cycling parameters: 96° 5 min, 35×[94° 1 min, 50° 1 min, 72° 2 min], 72° 10 min, followed by a hold at 4°.

To determine whether the PCR reaction amplified the correct gene region, 5 μl of the PCR product was run in an E-Gel (Invitrogen) cassette stained with SYBR-Safe dye alongside a 100 bp latter (Promega) for 15 minutes and then visualized using a blue-light transilluminator. This revealed a single band of approximately 700 base pairs in length.

To visualize the quality of the DNA, 5 μl of the PCR product was purified using the enzyme mixture EXO-SAPIT (USB Corp.) and heated in the PCR machine according to the manufacturers instructions. After purification, 2.5 μl of the purified PCR product was mixed with 0.5 μl of the forward primer or 0.5 μl of the reverse primer and 10 μl sterile water. The forward and reverse primer mixtures were then sequenced by Sanger Sequencing using an Applied Biosystems 3730 DNA Analyzer according to the manufacturer's protocol. The resulting DNA sequences showed multiple DNA templates (FIG. 2).

Separation of Multiple DNA Templates

In order to separate the multiple DNA templates, the unpurified PCR products were cloned into E. coli using the TOPO TA Cloning Kit (Invitrogen) following the manufacturers instructions, or in ¼, or ½ reactions. Bacteria were plated on kanamycin or ampicillin. After approximately 24 hours at 37° C., the plates were checked for growth and 25 colonies were transferred using sterile techniques to individual tubes containing TE buffer or water, or used directly in a subsequent PCR reaction using the primers M13 forward (5′-GTAAAACGACGGCCAG-3′) and reverse (5′-CAGGAAACAGCTATGAC-3′) to excise the ITS region from the bacteria and amplify it. The PCR parameters for this reaction are as follows: 94° 12 min, 30×[94° 1 min, 58° 1 min, 72° 2 min], 72° 7 min. The amplicons from this reaction were then visualized, purified, and sequenced as described in the previous paragraph.

Visual analysis revealed that only 20 of the 25 colonies contained inserts of the proper size. Sequencing revealed that 12 of these 20 colonies contained sequences similar to those of Schisandra species, while 8 contained sequences similar to Glycine max (soy). All of the Schisandra sequences were identical (S. chinensis 1, FIG. 3).

Sequences were compared initially to the database of authenticated reference materials and to GenBank using the NCBI Blast algorithm, National Center for Biotechnology Information (NCBI) and the National Library of Medicine (www.ncbi.nlm.gov/cgi-biniBLAST). All of the Schisandra sequences were identical to reference materials of S. chinensis.

The Schisandra sequences were added to a database, an aligned matrix was produced in SeaView containing only Schisandra sequences, and phylogenetic analysis was conducted using PAUP* using a ML algorithm. This analysis revealed that the Schinsandra sequences sampled are nested within other S. chinensis sequences with a 99% ML bootstrap value.

These results indicate that the disclosed methods were successful in identifying the primary species at the species level. In addition, the methods resulted in the identification of soy (Glycine max), a contaminant species not listed on the label for the schisandra powder capsule, and were successful in roughly quantifying the ratio of the primary and contaminating species. The combination of cloning to separate the DNA templates and then sequencing each resultant colony allowed for positive identification of the two components; without cloning, neither of the species would have been identified due to overlapping DNA signals.

### Example 2

### Genetic Analysis of Multi-Component Product

Genomic DNA Isolation

Total genomic DNA from two capsules produced by company B and labeled as containing more than 20 plant and fungal species was extracted using the QIAmp Stool Kit (Qiagen). The extraction was performed using the automated QIAcube machine (Qiagen) running the standard protocol for the detection of human pathogens with the QIAamp Stool Kit. Using sterile techniques, a total of 20 μl of genomic DNA was transferred using a pipet to a new sterile microcentrifuge tube and placed back into the QIAcube. The genomic DNA was purified using the MinElute PCR Purification Kit (Qiagen) and the standard protocol for this kit on the QIAcube. The purified DNA was removed from the QIAcube and used for PCR amplification.

PCR Amplification

DNA amplifications were carried out in a final volume of 25 μL, using 2.5 μL of DNA extract as template (Valentini 2009). The amplification mixture contained 1 U of AmpliTaq Gold DNA Polymerase (Applied Biosystems), 10 mm Tris-HCl, 50 mm KCl, 2 mm of MgCl₂, 0.2 mm of each dNTPs, 0.1 μm of each primer, and 0.005 mg of bovine serum albumin (BSA, Roche Diagnostics). After 10 min at 95° C. (Tag activation), the PCR cycles were as follows: 35 cycles of 30 s at 95° C., 30 s at 55° C.; the elongation was removed in order to reduce the +A artifact. Each sample was amplified with primers g and h (Taberlet 2006), modified by the addition of a specific tag on the 5′ end in order to allow the recognition of the sequences after pyrosequencing, where all the PCR products from the different samples are mixed together. These tags were composed of six nucleotides, always starting with CC on the 5′ end, followed by four variable nucleotides that were specific to each sample.

PCR products were purified using the MinElute PCR purification kit (Qiagen). DNA quantification was carried out using the NanoDrop ND-1000 UV-Vis Spectrophotometer (NanoDrop Technologies). A mix was then made taking into account these DNA concentrations in order to obtain roughly the same number of molecules per PCR product corresponding to the different samples. Samples were multiplexed using a previously disclosed protocol (Meyer 2008). Large-scale pyrosequencing was carried out on the 454 sequencing system (Roche) following manufacturer's instructions.

The unassembled DNA sequences provided by the 454 were compared to a database of reference sequences using the NCBI BLAST algorithm. Phylogenetic analysis for short DNA fragments was conducted as described previously using contigs that had been assembled into consensus sequences for each taxon (Krause 2008).

The two samples produced more than 60 Mbp of sequence data from over 600,000 sequences. When compared to a database using Blast, all of the species labeled on the container were identified to species level. In addition, more than ten contaminant species not listed on the label were identified, including bacteria, fungus, and plant contaminant species.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Krause et. al. Nucl Acids Res 36:2230 (2008)
2. Meyer et. al. Nature Protocols 3:267 (2008)
3. Taberlet et al. Nucl Acids Res 35:e14 (2006)
4. Teletchea et al. TRENDS Biotechnol 23:359-366 (2005)
5. Valentini et al. Mol Ecology Res 9:51-60 (2009)
6. White et al. PCR Protocols: A Guide to Methods and Applications, pp. 315-322, Innis et al. Eds. (1990)
7. Yip et al. Chinese Medicine 2:9 (2007)
8. Zhang et al. Food Drug Analysis 15:1 (2007)

What is claimed is:

1. A method for identifying component species in a natural product comprising:
a) isolating genomic DNA from a natural product;
b) amplifying one or more target regions from said genomic DNA to produce amplified target regions;
c) sequencing said amplified target regions to obtain component species sequences;
d) comparing said component species sequences to one or more reference sequences from primary species and/or contaminating species; and
e) classifying each component species sequence as a primary species sequence or a contaminating species sequence.

2. A method for identifying component species in a natural product comprising:
a) isolating genomic DNA from a natural product;
b) amplifying one or more target regions from said genomic DNA to produce amplified target regions;
c) separating said amplified target regions into multiple DNA templates by cloning said multiple DNA templates into one or more bacterial host cells;
d) isolating DNA from said bacterial host cells;
e) sequencing the DNA from said bacterial host cells to obtain component species sequences;
f) comparing said component species sequences to one or more reference sequences from primary species and/or contaminating species; and
g) classifying each component species sequence as a primary species sequence or a contaminating species sequence.

3. A method for identifying component species in a natural product comprising:
a) isolating genomic DNA from a natural product;
b) amplifying one or more target regions from said genomic DNA to produce amplified target regions;
c) performing simultaneous sequencing on the amplified target regions to obtain component species sequences;
d) comparing said component species sequences to one or more reference sequences from primary species and/or contaminating species; and
e) classifying each component species sequence as a primary species sequence or a contaminating species sequence.

4. The method of any of claims 1 to 3, wherein sequencing is carried out using Sanger sequencing.

5. The method of any of claims 1 to 3, wherein sequence is carried out using a next-generation sequencing technique selected from the group consisting of Solexa, SOliD, Ion Torrent, and PyroMark or 454 Pyrosequencing.

6. The method of any of claims 1 to 3, further comprising analyzing single nucleotide polymorphisms from said component species sequences.

7. The method of any of claims 1 to 3, further comprising conducting a phylogenetic analysis of said component species sequences.

8. The method of any of claims 1 to 3, wherein genomic DNA is purified prior to amplification.

9. The method of any of claims 1 to 3, wherein amplified DNA is purified prior to sequencing.


SEQUENCE LISTING (16 sequences)

SEQ ID NO: 1 - DNA, 22 bp, Artificial Sequence
  PCR primer
ggaagtaaaagtcgtaacaagg

SEQ ID NO: 2 - DNA, 20 bp, Artificial Sequence
  PCR primer
tcctccgcttattgatatgc

SEQ ID NO: 3 - DNA, 16 bp, Artificial Sequence
  PCR primer
gtaaaacgacggccag

SEQ ID NO: 4 - DNA, 17 bp, Artificial Sequence
  PCR primer
caggaaacagctatgac

SEQ ID NO: 5 - DNA, 39 bp, Schisandra chinensis
ccttgtatgacacgccttggtcttgtgccatgcgttgat

SEQ ID NO: 6 - DNA, 39 bp, Schisandra spenanthera
ccttgtatgacatgctttggtcttgtgccatgcgttgat

SEQ ID NO: 7 - DNA, 40 bp, Schisandra bicolor
ccttgtttgacaacgctttggtcttgtgccatgcgttgat

SEQ ID NO: 8 - DNA, 39 bp, Schisandra glabra
ccttgtttgacacgctttggtcttgtgccatgcgttgat

SEQ ID NO: 9 - DNA, 39 bp, Schisandra glaucescens; identical to SEQ ID NO: 6

SEQ ID NO: 10 - DNA, 39 bp, Schisandra grandiflora; identical to SEQ ID NO: 6

SEQ ID NO: 11 - DNA, 39 bp, Schisandra henreyi
ccttgtatgacacgctttggtcttgtgccatgcgttgat

SEQ ID NO: 12 - DNA, 39 bp, Schisandra plena
ccttgtttgacacgctttggtctcgtgccatgcgttgat

SEQ ID NO: 13 - DNA, 39 bp, Schisandra propinqua
ccttatttgacacgctttggtctcgtgccatgcgttgat

SEQ ID NO: 14 - DNA, 39 bp, Schisandra pubescens; identical to SEQ ID NO: 11

SEQ ID NO: 15 - DNA, 39 bp, Schisandra rubrifolia; identical to SEQ ID NO: 6

SEQ ID NO: 16 - DNA, 39 bp, Schisandra viridis; identical to SEQ ID NO: 6